SEQUENCE LISTING (8 sequences)

SEQ ID NO: 1 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 107 aa, synthetic construct [sequence omitted]

SEQ ID NO: 6 - protein, 119 aa, synthetic construct [sequence omitted]

SEQ ID NO: 7 - protein, 107 aa, synthetic construct [sequence omitted]

SEQ ID NO: 8 - protein, 120 aa, synthetic construct [sequence omitted]

(19) United States
(12) Patent Application Publication
(10) Pub. No.: US 2025/0177364 A1
(43) Pub. Date: Jun. 5, 2025

(54) COMBINATION THERAPIES FOR BREAST CANCER

(71) Applicants: Genentech, Inc., South San Francisco, CA (US); Hoffmann-La Roche Inc., Little Falls, NJ (US)

(72) Inventors: Daniel Eiger, Basel (CH); Marc Antoine Hafner, Menlo Park, CA (US); Sarah Louise Heeson, Arlesey, Bedfordshire (GB); Ciara Metcalf, Kensington, CA (US); Mina Nikanjam, La Jolla, CA (US); Raf Poppe, Basel (CH); Eleonora Restuccia, Basel (CH); Mahesh Ratanlal Shivhare, Cheshunt, Hertfordshire (GB); Jing Zhu, Moraga, CA (US)

(73) Assignees: Genentech, Inc., South San Franciso, CA (US); Hoffmann-La Roche Inc., Little Falls, NJ (US)

(21) Appl. No.: 18/844,654

(22) PCT Filed: Mar. 10, 2023
(86) PCT No.: PCT/US2023/064143

§ 371 Date: Sep. 6, 2024

Related U.S. Application Data
(60) Provisional application No. 63/269,326, filed on Mar. 14, 2022.

(51) Int. Cl. A61K 31/437; A61K 31/337; A61K 31/506; A61K 31/519; A61K 39/00; A61P 35/00; C07K 16/32

(52) U.S. Cl. CPC A61K 31/437; A61K 31/337; A61K 31/506; A61K 31/519; A61P 35/00; C07K 16/32; A61K 2039/507; A61K 2039/545

(57) ABSTRACT

The present application discloses combinations for treating estrogen receptor-positive and HER2-positive breast cancer patients. The combinations comprise pertuzumab and trastuzumab (e.g. a fixed dose combination of pertuzumab and trastuzumab, PH FDC) plus giredestrant. In one embodiment, the combination further includes a CDK4/6 inhibitor, such as abemaciclib or palbociclib.

### CLAIM OF PRIORITY

This application claims benefit of priority to U.S. provisional patent application Ser. No. 63/269,326, filed Mar. 14, 2022, the contents of which are incorporated by reference herein in their entirety.

### SEQUENCE LISTING

This application contains a Sequence Listing which has been submitted electronically in XML format and is hereby incorporated by reference in its entirety. Said XML copy, created on Feb. 22, 2023, is named P37389-WO.xml and is 12,228 bytes in size.

### FIELD OF THE INVENTION

The invention concerns combination therapies for estrogen receptor-positive and HER2-positive breast cancer patients. The combinations comprise pertuzumab and trastuzumab (e.g. a fixed dose combination of pertuzumab and trastuzumab, PH FDC) plus giredestrant, and optionally further comprise a CDK4/6 inhibitor (e.g. abemaciclib or palbociclib).

### BACKGROUND OF THE INVENTION

### Pertuzumab (PERJETA®)

Pertuzumab is a HER2/neu receptor antagonist administered by intravenous infusion for:
Use in combination with trastuzumab and docetaxel for treatment of patients with HER2-positive metastatic breast cancer (MBC) who have not received prior anti-HER2 therapy or chemotherapy for metastatic disease.
Use in combination with trastuzumab and chemotherapy as:
   neoadjuvant treatment of patients with HER2-positive, locally advanced, inflammatory, or early stage breast cancer (either greater than 2 cm in diameter or node positive) as part of a complete treatment regimen for early breast cancer.
   adjuvant treatment of patients with HER2-positive early breast cancer at high risk of recurrence.

### Trastuzumab (HERCEPTIN®)

Trastuzumab is a HER2/neu receptor antagonist administered by intravenous infusion for:
The treatment of HER2-overexpressing breast cancer.
The treatment of HER2-overexpressing metastatic gastric or gastroesophageal junction adenocarcinoma.

### Recombinant Hyaluronidase Human Injection (HYLENEX®)

Recombinant hyaluronidase human injection is a tissue permeability modifier administered by subcutaneous fluid administration used:
in subcutaneous fluid administration for achieving hydration
to increase the dispersion and absorption of other injected drugs
in subcutaneous urography for improving resorption of radiopaque agents

### Trastuzumab and Hyaluronidase-Oysk (HERCEPTIN HYLECTA™)

Trastuzumab and hyaluronidase-oysk is a combination of trastuzumab, a HER2/neu receptor antagonist, and recombinant human hyaluronidase, an endoglycosidase, indicated in adults for:
The treatment of HER2-overexpressing breast cancer.

### Pertuzumab and Trasutuzumab Fixed Dose Combination (PH FDC. PHESGO™)

PH FDC (PHESGO™) is a combination of pertuzumab and trastuzumab, HER2/neu receptor antagonists, and hyaluronidase, an endoglycosidase, indicated for:

Use in combination with chemotherapy as:
neoadjuvant treatment of patients with HER2-positive, locally advanced, inflammatory, or early stage breast cancer (either greater than 2 cm in diameter or node positive) as part of a complete treatment regimen for early breast cancer.
adjuvant treatment of patients with HER2-positive early breast cancer at high risk of recurrence.

Use in combination with docetaxel for treatment of patients with HER2-positive metastatic breast cancer (MBC) who have not received prior anti-HER2 therapy or chemotherapy for metastatic disease.

PH FDC SC is disclosed in US 2018/0296470 A1, US 2021/0403599 A1, and WO 2022/013189 A1.

### Ado-Trastuzumab Emtansine

Ado-trastuzumab emtansine for injection, for intravenous use is a HER2-targeted antibody and microtubule inhibitor conjugate indicated, as a single agent, for:
the treatment of patients with HER2-positive, metastatic breast cancer who previously received trastuzumab and a taxane, separately or in combination. Patients should have either:
   received prior therapy for metastatic disease, or
   developed disease recurrence during or within six months of completing adjuvant therapy.
the adjuvant treatment of patients with HER2-positive early breast cancer who have residual invasive disease after neoadjuvant taxane and trastuzumab-based treatment.

### Giredestrant

Giredestrant is a potent, orally bioavailable ER-antagonist and inducer of ER-degradation that competes with estrogens for binding to the ER with low nanomolar potency. It is being developed as a new endocrine therapy (ET) for the treatment of patients with ER-positive advanced breast cancer (ABC), as well as early breast cancer (EBC) (Liang et al. J Med Chem. 64:11841-56 (2021)). Giredestrant has higher potency compared to fulvestrant, tamoxifen and other oral SERDs under development (Liang et al.).

Giredestrant antagonizes the effects of estrogens via competitive binding to the ligand-binding domain (LBD) of both wild-type and mutant ER with nanomolar potency. Upon binding, giredestrant induces an inactive conformation to the ER LBD, as measured by displacement of co-activator peptides. In addition to its direct antagonist properties, the mechanism of action of giredestrant includes reducing levels of ER protein through proteasome-mediated degradation. Degradation of ER is hypothesized to enable full suppression of ER signaling, which is not achieved by first-generation ER therapeutics such as tamoxifen that display partial agonism. Giredestrant potently inhibits the proliferation of multiple ER-positive BC cell lines in vitro, including cells engineered to express clinically relevant mutations in ER.

In a Phase I study (Study GO39932), giredestrant monotherapy showed promising signs of clinical activity at the recommended phase 2 dose of 30 mg daily and was safe in patients with previously treated ER-positive, HER2-negative ABC (Jhaveri et al. J Clin Oncol 39(15 suppl):1017 (2021)).

In the Phase II, randomized coopERA study (study WO42133), neoadjuvant giredestrant was demonstrated to be superior to anastrozole to achieve Ki67 suppression and complete cell cycle arrest in patients with ER-positive, HER2-negative EBC (Hurvitz ct al. Ann Oncol 2021; 32:S1285-6(2021)).

Studies evaluating endocrine therapy (ET) in combination with anti-HER2 antibody therapy include: Witters et al. Breast Cancer Res Treat. 42:1-5 (1997); Kunisue et al. Br J Cancer. 82:46-51 (2000); Kaufman et al. J Clin Oncol. 27:5529-37 (2009); Leary et al. Clin Cancer Res. 16:1486-97 (2010); Schwartzberg et al. Oncologist. 15 (2):122-9 (2010) Erratum in: Oncologist. 15 (3):327 (2010); Wang et al. Breast Cancer Res. 13 (6): 121 (2011); Huober et al. Breast. 21:27-33 (2012); Rimawi et al. J Clin Oncol. 36:2826-35 (2018); and Metzger et al. Cancer Res 79 (4_Supplement): OT3-02-07 (2019). These studies did not evaluate safety and efficacy of giredestrant, a novel oral selective estrogen receptor degrader (SERD), in combination with pertuzumab and trastuzumab.

NCT04802759 (First posted: Mar. 17, 2021; Last Update Posted: Feb. 7, 2022) is a Phase Ib/II, open-label, multicenter, randomized umbrella study in participants with breast cancer. Cohort 1 will focus on participants with inoperable, locally advanced or metastatic, estrogen receptor (ER)-positive, HER2-negative breast cancer who had disease progression during or following treatment with a cyclin-dependent kinase 4/6 inhibitor (CDK4/6i; e.g., palbociclib or abemaciclib) in the first- or second-line setting. Cohort 2 will focus on inoperable, locally advanced or metastatic, ER-positive, HER2-positive breast cancer with prior disease progression on trastuzumab-and-taxane- and ado-trastuzumab emtansine-based therapies. Cohort 2 includes arms evaluating: giredestrant+PH FDC, giredestrant+PH FDC+abemaciclib, and giredestrant+PH FDC+palbociclib.

Therapies for ER-positive. HER2-positive Breast Cancer

ER expression in HER2-positive BC implies a rather distinct biology compared to that of ER-negative, HER2-positive BC: patients diagnosed with ER-positive, HER2-positive BC have tumors that are less proliferative, have lower HER2 gene amplification, and lower response rates to chemotherapy with anti-HER2 therapies. Baselga et al. N Engl J Med. 366:109-19 (2012); Gianni et al. Lancet Oncol. 13:25-32 (2012); Schneeweiss et al. Ann Oncol. 24:2278-84. Epub 2013 May 22; Loi et al. JAMA Oncol. 2:1040-7 (2016).

A bi-directional cross-talk between the HER-family and ER has been fully characterized at cellular level, whereby suppression of either receptor alone is associated with upregulation of the other, ultimately leading to resistance to therapy (Cortés et al. Nat Rev Clin Oncol. 8:307-11 (2011)). PERTAIN (Study MO27775) also demonstrated the beneficial effect of a dual HER2 blockade with ET; an improvement in progression-free survival (PFS) alongside good tolerability was observed with the addition of pertuzumab to trastuzumab (PH) plus an aromatase inhibitor (AI), over trastuzumab plus AI alone (Rimawi et al. J Clin Oncol. 2018; 36:2826-35 (2018)).

ER-positive, HER2-positive patients with ABC are more frequently identified as having luminal BC subtypes compared to ER-negative, HER2-positive disease, which relies more intensely on the ER pathway, and frequently experience an intrinsic molecular subtype shifting/increased predominance from HER2-enriched to luminal BC upon chemotherapy+anti-HER2 therapy exposure (Perou et al. Nature. 406:747-52 (2000); Carey et al. J Clin Oncol. 2016; 34:542-9. Epub 2015 Nov. 2; Cejalvo et al. Ann Oncol. 28 (suppl_5): v595-v604 (2017); Brasó-Maristany et al. Nat Commun. 11:385 (2020).

Patients with ER-positive, HER2-positive BC have been shown in exploratory analyses of a phase 3 trial to benefit from enhanced endocrine therapy partners with trastuzumab and lapatinib. Lambertini et al. Breast Cancer Res Treat. 177:103-14. Epub 2019 May 27.

Available maintenance therapies for ER-positive, HER2-positive ABC, fulvestrant and anastrozole, are reported in: Robertson et al. Breast Cancer Res Treat. 136:503-11. Epub 2012 Oct. 13; Ellis et al. J Clin Oncol. 33:3781-7 (2015); and Robertson et al. Lancet. 388:2997-3005 (2016).

Despite advances in early diagnosis and curative multimodality treatments, some patients may still experience a metastatic recurrence or present with “de novo” metastatic breast cancer (MBC). In this setting, the main goals of treatment are to improve the quality of life and prolong patient survival as there still is not a cure (Cardoso et al. Ann Oncol. 31:1623-49 (2020)). Thus there continues to be a need for treatments with better benefit/risk profiles that prolongs PFS and other survival endpoints of patients with ER-positive, HER2-positive ABC.

### SUMMARY OF THE INVENTION

In one aspect, the invention concerns a method of treating an estrogen receptor-positive and HER2-positive breast cancer patient comprising administering to the patient a combination of pertuzumab, trastuzumab, and giredestrant in amounts effective to treat the breast cancer.

In another aspect, the invention concerns a method of treating an estrogen receptor-positive and HER2-positive breast cancer patient with advanced breast cancer comprising administering to the patient
a. induction therapy comprising four to eight cycles of a combination of pertuzumab and trastuzumab fixed dose combination (PH FDC) and docetaxel or paclitaxel in amounts effective to treat the breast cancer, followed by:
b. maintenance therapy comprising a combination of giredestrant and PH FDC in amounts effective to treat the breast cancer.

In yet another aspect, the invention concerns a method of treating an estrogen receptor-positive and HER2-positive breast cancer patient with advanced breast cancer comprising administering to the patient a combination of pertuzumab and trastuzumab fixed dose combination (PH FDC), giredestrant, and palbociclib in amounts effective to treat the advanced breast cancer.

In a further aspect, the invention provides a method of treating an estrogen receptor-positive and HER2-positive breast cancer patient with advanced breast cancer comprising administering to the patient a combination of pertuzumab and trastuzumab fixed dose combination (PH FDC), giredestrant, and abemaciclib in amounts effective to treat the advanced breast cancer.

In certain embodiments of any of the above methods:
the pertuzumab and trastuzumab are administered as a fixed dose combination of pertuzumab and trastuzumab (PH FDC);
the breast cancer is advanced breast cancer, including locally advanced unresectable breast cancer and/or metastatic breast cancer;
treatment with the combination is more effective than treatment with pertuzumab and trastuzumab without giredestrant or than treatment with PH FDC and palbociclib or abemaciclib without giredestrant;
the combination is administered as a maintenance therapy after induction therapy;
the combination further comprises CDK4/6 inhibitor, e.g. palbociclib or abemaciclib, e.g. where the patient has progressed on prior HER2-therapy, e.g. where the patient had prior disease progression on trastuzumab treatment and on HER2-antibody drug conjugate (ADC) treatment (including ado-trastuzumab emtansine ADC or trastuzumab deruxtecan ADC, for example).

### BRIEF DESCRIPTION OF THE DRAWINGS

FIGS. 1A and 1B show the amino acid sequences of pertuzumab light chain (FIG. 1A; SEQ ID NO. 1) and heavy chain (FIG. 1B; SEQ ID NO. 2). CDRs are shown in bold. The carbohydrate moiety is attached to Asn 299 of the heavy chain. Boundaries of the variable light domain (SEQ ID NO: 5) and variable heavy domain (SEQ ID NO: 6) are indicated by arrows.

FIGS. 2A and 2B show the amino acid sequences of trastuzumab light chain (FIG. 2A; SEQ ID NO. 3) and heavy chain (FIG. 2B; SEQ ID NO. 4), respectively. Boundaries of the variable light domain (SEQ ID NO: 7) and variable heavy domain (SEQ ID NO: 8) are indicated by arrows.

FIG. 3 depicts in vitro anti-proliferative activity of ER inhibition by giredestrant, and/or HER2 inhibition by the combination of trastuzumab and pertuzumab, assessed using the growth rate (GR) method in Example 1. Growth rate (GR) under control conditions is set to 1; lower growth rates equate to a greater anti-proliferative effect. Individual points reflect GR values at 0.3 μM giredestrant, and at 30 μM trastuzumab and pertuzumab.

FIG. 4 shows Study Schema for the PH FDC+giredestrant±CDK4/6 inhibitor combination therapy trial in Example 2 (Morpheus Breast).

FIG. 5 shows Study Schema for the PH FDC and giredestrant combination therapy trial in Example 3 (heredERA). ABC=advanced breast cancer; AI=aromatase inhibitor; CR=complete response; ER-estrogen receptor; ET=endocrine therapy; LHRHa=luteinizing hormone-releasing hormone agonists; LVEF=left ventricular ejection fraction; R=randomized; PD-progressive disease; SD=stable disease. Docetaxel or paclitaxel at the investigator's discretion as per the standard of care. Patient must receive a minimum of four complete cycles of induction therapy (including cycles received prior to study enrollment, if applicable). At the investigator's discretion, participants who tolerate six induction therapy cycles well and do not experience progressive discasc (PD) may be given up to two additional cycles, up to a maximum of eight cycles. Optional endocrine therapy (ET) of investigator's choice is allowed based on the standard of care (ET can include an aromatase inhibitor or tamoxifen±LHRHa or gonadal ablation). Pre- and perimenopausal women, and all men will receive a LHRHa every 28 days and up to 28 days prior to the first giredestrant dose. Alternatively, women who are pre- or perimenopausal can be treated with bilateral oophorectomy.

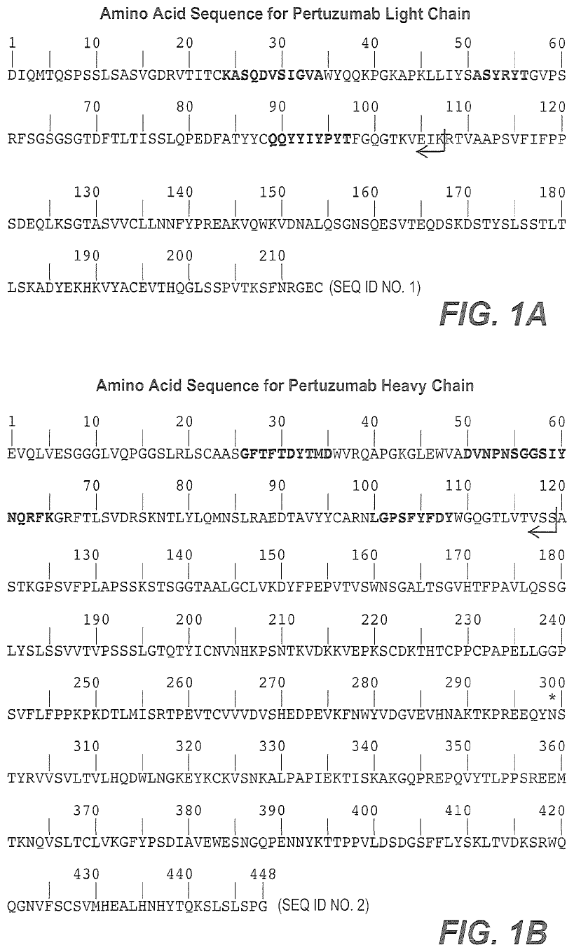

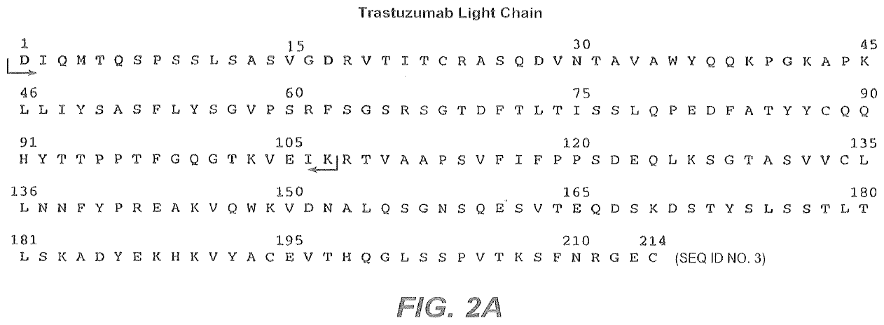

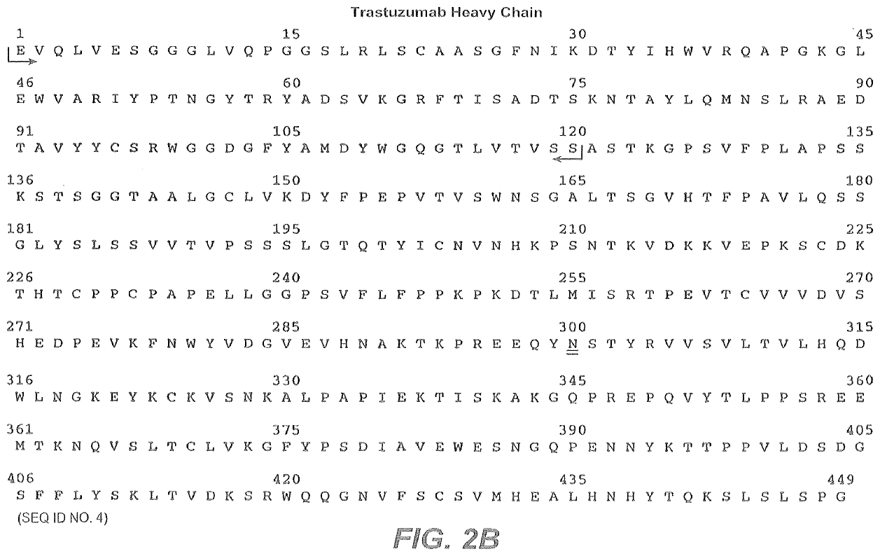

### DETAILED DESCRIPTION OF THE PREFERRED EMBODIMENTS

### I. Definitions

Abbreviations

| | |
|---|---|
| ABC | advanced breast cancer |
| ADA | anti-drug antibody |
| AI | aromatase inhibitor |
| BC | breast cancer |
| CBR | clinical benefit rate |
| CHF | congestive heart failure |
| CR | complete response |
| DCR | disease control rate |
| DOR | duration of response |
| ECHO | Echocardiogram |
| ECOG | Eastern Cooperative Oncology Group |
| ER | estrogen receptor |
| ET | endocrine therapy |
| FFPE | formalin-fixed, paraffin-embedded |
| GHS/QoL | global health status/ quality of life |
| HER2 | HER2, human epidermal growth factor |
| | receptor 2 |
| HR | Hazard ratio |
| IHC | immunohistochemistry |
| ISH | in situ hybridization |
| LHRHa | luteinizing hormone-releasing hormone |
| | agonist |
| LVEF | left ventricular ejection fraction |
| LVSD | left ventricular systolic dysfunction |
| MBC | metastatic breast cancer |
| MUGA | multiple-gated acquisition |
| NCI CTCAE | National Cancer Institute Common |
| | Terminology Criteria for Adverse Events |
| NYHA | New York Heart Association |
| ORR | objective response rate |
| OS | overall survival |
| PD | progressive disease |
| PFS | progression-free survival |
| PH | pertuzumab and trastuzumab |
| PK | pharmacokinetic |
| PO | by mouth; orally |
| PR | partial response |
| Q3W | every 3 weeks |
| QD | every day |
| RECIST | Response Evaluation Criteria in Solid Tumors |
| SARS-CoV-2 | severe acute respiratory syndrome |
| | coronavirus 2 |
| SC | subcutaneous |
| SD | stable disease |
| SERD | selective estrogen receptor degrader |
| SERM | selective estrogen receptor modulators |

For the purposes herein, “pertuzumab” refers to an antibody comprising variable light amino acid sequence of SEQ ID NO: 5 and variable heavy amino acid sequence of SEQ ID NO: 6. In one embodiment, pertuzumab comprises an intact IgG1 antibody. In one embodiment, pertuzumab comprises the light chain amino acid sequence in SEQ ID NO: 1 and heavy chain amino acid sequence in SEQ ID NO: 2. In one embodiment, pertuzumab is produced by recombinant Chinese Hamster Ovary (CHO) cells.

For the purposes herein, “trastuzumab” refers to an antibody comprising variable light amino acid sequence of SEQ ID NO: 7 and variable heavy amino acid sequence of SEQ ID NO: 8. In one embodiment, trastuzumab comprises an intact IgG1 antibody. In one embodiment, the trastuzumab comprises the light chain amino acid sequence of SEQ ID NO: 3 and the heavy chain amino acid sequence of SEQ ID NO: 4. In one embodiment, trastuzumab is produced by Chinese Hamster Ovary (CHO) cells.

“Pertuzumab and trastuzumab fixed dose combination” or “PH FDC” as used herein refer to a ready-to-use co-formulation comprising a fixed dose of pertuzumab and a fixed dose of trastuzumab and, optionally, recombinant human hyaluronidase (rHuPH20). The PH FDC is administered subcutaneously.

A “loading dose FDC” refers to an initial dose FDC comprising 1200 mg pertuzumab and 600 mg trastuzumab, and, optionally, 30,000 units of rHuPH20. An exemplary loading dose formulation comprises: pertuzumab (dose: 1200 mg; concentration: 80 mg/mL); trastuzumab (dose: 600 mg; concentration: 40 mg/mL); rHuPH20 (concentration: 2000 U/mL); pH: 5.5; 20 mM L-histidine/HCl; trehalose: 70 mM; sucrose: 133 mM; polysorbate 20 (PS20): 0.04% (0.4 mg/mL); 10 mM methionine; nominal fill volume 15 mL; vial: 20 mL/20 mm.

A “maintenance dose FDC” refers to a maintenance dose FDC which comprises 600 mg pertuzumab and 600 mg trastuzumab, and, optionally, 20,000 units of rHuPH20. An exemplary maintenance dose formulation comprises: pertuzumab (dose: 600 mg; concentration: 60 mg/mL); trastuzumab (dose: 600 mg; concentration: 60 mg/mL); rHuPH20 (concentration: 2000 U/mL); pH: 5.5; 20 mM L-histidine/HCl; trehalose: 105 mM; sucrose: 100 mM; polysorbate PS20:0.04% (0.4 mg/mL); 10 mM methionine; nominal fill volume: 10 mL; vial: 15 mL/20 mm.

“Endocrine therapy” or “ET” refers to therapy that slows or stops the growth of hormone-sensitive tumors by blocking the body's ability to produce hormones or by interfering with effects of hormones on breast cancer cells. Examples of ET drugs include: aromatase inhibitors (AIs), such as anastrozole, exemestane, and letrozole; selective estrogen receptor modulators (SERMs), such as tamoxifen, raloxifene, toremifene, and giredestrant; estrogen receptor antagonists, such as fulvestrant, toremifene, and giredestrant; luteinizing hormone-releasing hormone agonist (LHRHa), such as goserelin acetate, leuprolide acetate, triptorelin pamoate. A preferred ET herein is giredestrant.

For the purposes herein, “giredestrant” or “GDC-9545” refer to a compound having the structure:

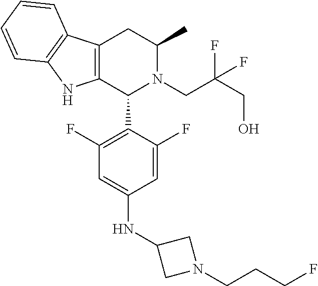 [SMILES: C[C@@H]1Cc2c([nH]c3ccccc23)[C@@H](c2c(F)cc(NC3CN(CCCF)C3)cc2F)N1CC(F)(F)CO]

having the chemical name 3-((1R,3R)-1-(2,6-difluoro-4-((1-(3-fluoropropyl) azetidin-3-yl) amino)phenyl)-3-methyl-1,3,4,9-tetrahydro-2H-pyrido[3,4-b]indol-2-yl)-2,2-difluoropropan-1-ol, including a pharmaceutically acceptable salt thereof. In one embodiment, giredestrant is a tartrate salt. “Giredestrant” as used herein refers to free base and pharmaceutically acceptable salts of giredestrant including a tartrate salt thereof. Giredestrant is also known as GDC-9545.

A “taxane” is a chemotherapy which inhibits mitosis and interferes with microtubules. Examples of taxanes include paclitaxel (TAXOL®; Bristol-Myers Squibb Oncology, Princeton, N.J.); cremophor-free, albumin-engineered nanoparticle formulation of paclitaxel or nab-paclitaxel (ABRAXANE™, American Pharmaceutical Partners, Schaumberg, Illinois); and docetaxel (TAXOTERE®; Rhône-Poulenc Rorer, Antony, France). In one embodiment, the taxane is paclitaxel. In one embodiment, the taxane is docctaxcl.

A “CDK4/6 inhibitor” is a cyclin-dependent kinase (CDK) inhibitor that targets the CDK4 and/or CDK6 cell cycle pathway. Exemplary such inhibitors include: abemaciclib (VERZENIO®), palbociclib (IBRANCE®), and ribociclib (KISQALI®).

“Abemaciclib” specifically inhibits CDK4 and CDK6 and comprises the chemical structure: 2-pyrimidinamine, N-(5-((4-ethyl-1-piperazinyl)methyl)-2-pyridinyl)-5-fluoro-4-(4-fluoro-2-methyl-1-(1-methylethyl)-1H-benzimidazol-6-yl).

“Palbociclib” selectively inhibits CDK4 and CDK6 and comprises the chemical structure: 6-acetyl-8-cyclopentyl-5-methyl-2-((5-(piperazin-1-yl) pyridin-2-yl)amino)pyrido[2,3-d]pyrimidin-7 (8H)-one.

The term “cancer” refers to the physiological condition in mammals that is characterized by unregulated cell growth.

An “advanced” cancer is one which has spread outside the site or organ of origin, either by local invasion (“locally advanced”) or metastasis (“metastatic”). Accordingly, the term “advanced” cancer includes both locally advanced and metastatic disease.

“Locally advanced breast cancer” or “LABC” refers to cancer that has spread from where it started in the breast to nearby tissue or lymph nodes, but not to other parts of the body. In one embodiment, the LABC is unresectable.

“Metastatic breast cancer” or “MBC” refers to cancer that has spread from the breast to other parts of the body, such as the bones, liver, lungs, or brain. Metastatic breast cancer may also be referred to as stage IV breast cancer.

“Early-stage breast cancer” or “EBC” herein refers to breast cancer that has not spread beyond the breast or the axillary lymph nodes. Such cancer is generally treated with neoadjuvant or adjuvant therapy.

“Ncoadjuvant therapy” or “ncoadjuvant treatment” or “ncoadjuvant administration” refers to systemic therapy given prior to surgery.

“Adjuvant therapy” or “adjuvant treatment” or “adjuvant administration” refers to systemic therapy given after surgery.

Herein, a “patient” or “subject” is a human patient. The patient may be a “cancer patient,” i.e. one who is suffering or at risk for suffering from one or more symptoms of cancer, in particular breast cancer.

A “HER2-positive” cancer comprises cancer cells which have higher than normal levels of HER2. In one embodiment, HER2-positive cancer has an immunohistochemistry (IHC) score of 2+ or 3+ and/or is in situ hybridization (ISH), fluorescent in situ hybridization (FISH) or chromogenic in situ hybridization (CISH) positive, e.g. has an ISH/FISH/CISH amplification ratio of ≥2.0.

An “estrogen receptor positive” or “ER-positive” patient has a protein (receptor) that binds to the hormone estrogen on cancer cells in the patient. Cancer cells that are estrogen receptor positive may need estrogen to grow. These cells may stop growing or die when treated with substances that block the binding and actions of estrogen. In one embodiment, the patient with ER-positive tumor has ≥1% of tumor cells staining positive for ER, e.g. according to the American Society of Clinical Oncology/College of American Pathologists (ASCO/CAP) guidelines. In one embodiment the ER-positive patient also has HER2-positive cancer, and the ER-positivity is based on the same lesion that was used to determine HER2-positivity.

A patient who has “had prior disease progression on trastuzumab treatment and on HER2-ADC treatment” is one who, prior to treatment with a combinations therapy disclosed herein, has experienced disease progression while getting standard of care anti-HER2 therapy, for example: first line therapy comprising trastuzumab-therapy (e.g. trastuzumab-and-taxane-based systemic therapy, including in the early setting if recurrence occurred within 6 months of finishing adjuvant therapy) and of second line therapy comprising HER2 antibody drug conjugate (ADC) treatment (e.g. ado-trastuzumab emtansine or trastuzumab deruxtecan treatment).

“Induction therapy” refers to the first systemic treatment given for advanced or metastatic breast cancer from first diagnosis. In one embodiment, the induction therapy comprises treatment with PH FDC+taxane. In one embodiment, induction therapy will result in a minimum of stable disease (SD) in the patient, i.e. the patient does not have progressive disease (PD)

“Maintenance therapy” refers to treatment that is given to keep and optionally improve upon the benefit obtained with induction therapy. In one embodiment, the maintenance treatment comprises treatment with PH FDC plus giredestrant. In one embodiment, the maintenance therapy maintains stable disease. In one embodiment, maintenance therapy extends progression free survival (PFS) more than PH FDC alone.

“Treatment” refers to treatment with a drug or combination of drugs (e.g. PH FDC and giredestrant±CDK4/6 inhibitor) that achieves one or more efficacy endpoint(s) in the treatment of breast cancer in a patient. For the combination therapy disclosed herein with PH FDC and giredestrant, in one embodiment, the treatment achieves one or more efficacy endpoint(s) superior to that achieved with PH FDC alone. In one embodiment, the treatment achieves one or more efficacy endpoint(s) superior to that achieved with giredestrant alone. In one embodiment, the treatment achieves one or more efficacy endpoint(s) superior to that achieved with PH FDC alone and with giredestrant alone.

“Efficacy endpoint” refers to an event or outcome that can be measured objectively to determine whether the intervention being studied is beneficial. Exemplary efficacy endpoints herein include:
1. progression free survival (PFS),
2. overall survival (OS),
3. overall response rate (ORR),
4. duration of response (DOR),
5. disease control rate (DCR),
6. clinical benefit rate (CBR),
7. mean changes from baseline score in function (role, physical) and HRQOL by cycle and between treatment arms as assessed through the use of the Functional and GHS/QOL scales of the EORTC QLQ-C30. The treatment may result in 1, 2, 3, 4, 5, 6, or 7 of these endpoints superior to treatment with PH FDC alone or superior to treatment with PH FDC+giredestrant without CDK4/6 inhibitor.

“Progression free survival” or “PFS” is defined as the time from randomization (for example from the start of treatment or from the start of maintenance therapy) to the first occurrence of disease progression or death from any cause (whichever occurs first). In one embodiment, PFS is assessed according to RECIST v1.1 (Eisenhauer et al. European J. Cancer 45:228-247 (2009)).

“Overall survival” or “OS” is defined as the time from randomization (or from the start of treatment) to death from any cause.

“Overall response rate” or “ORR” is defined as the proportion of participants with a complete response (CR) or partial response (PR) on two consecutive occasions ≥4 weeks apart. In one embodiment, CR and PR are assessed according to RECIST v1.1.

“Duration of response” or “DOR” is defined as the time from the first occurrence of a documented objective response to disease progression or death from any cause (whichever occurs first). In one embodiment, DOR is assessed according to RECIST v1.1.

“Disease control rate” or “DCR is defined as proportion of patients with stable disease for ≥12 weeks or a complete or partial response, as determined by the investigator according to RECIST v1.1.

“Clinical Benefit Rate” or “CBR” is defined as the proportion of participants with stable disease (SD) for ≥24 weeks or a CR or PR. In one embodiment, CBR is assessed according to RECIST v1.1.

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

An “initial” or “loading” dose herein generally comprises an initial dose of a therapeutic agent administered to a patient, and is followed by one or more maintenance dose(s) thereof. Generally, a single loading dose is administered, but multiple loading doses are contemplated herein. In one embodiment, the loading dose exceeds the maintenance dose, so as to achieve the desired steady-state concentration of the therapeutic agent earlier than can be achieved with the maintenance dose. An exemplary loading dose for subcutaneous pertuzumab is 1200 mg. An exemplary loading dose for subcutaneous trastuzumab is 600 mg.

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks. An exemplary maintenance dose for subcutaneous pertuzumab is 600 mg. An exemplary maintenance dose for subcutaneous trastuzumab is 600 mg.

An “administration period” or “cycle” refers to a period of time comprising administration of one or more agents described herein (e.g. PH FDC, giredestrant, abemaciclib, and/or palbociclib) and an optional period of time comprising no administration of one or more of the agents described herein. For example, a cycle can be 21 days in total length with no rest period, or 28 days in total length and include administration of one or more agents for 21 days and a rest period of 7 days. A “rest period” refers to a period of time where at least one of the agents described herein (e.g. palbociclib) is not administered.

A “dosing regimen” refers to a period of administration of the agents described herein comprising one or more cycles, where each cycle can include administration of the agents described herein at different times or in different amounts.
“QD” refers to administration of a compound once daily.
“BID” (bis in die) refers to administration of a compound twice daily.
“PO” (per os) refers to oral administration of an agent described herein.
“SC” refers to subcutaneous administration of agent(s) described herein.

A “vial” is a container suitable for holding a liquid or lyophilized preparation. In one embodiment, the vial is a single-use vial, e.g. a 10 ml or a 20 ml single-use vial with a stopper, such as a 10 ml single use glass vial with a 20 mm stopper.

A “package insert” is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

Administration “in combination” encompasses combined administration and separate administration, in which case, administration of one therapeutic agent can occur prior to, simultaneously, and/or following, administration of another therapeutic agents. Thus, administration of FDC and giredestrant (and, optionally, CDK4/6 inhibitor) in combination encompasses combined administration and separate administration in either order.

A drug that is administered “concurrently” with one or more other drugs is administered during the same treatment cycle, on the same day of treatment as the one or more other drugs, and, optionally, at the same time as the one or more other drugs. For instance, for cancer therapies given every 3-weeks, the concurrently administered drugs are each administered on at least Day-1 of a 3-week cycle.

A “subcutaneous administration device” refers to a device able to administer a FDC as disclosed herein by subcutaneous administration to a patient. Exemplary devices contemplated herein include: a syringe, an injection device, an infusion pump, an injector pen, a needleless device, an autoinjector, and a subcutaneous patch delivery system. In one embodiment, the device is a hand-held syringe, e.g. comprising a 25G-27G (⅜″-⅝″) hypodermic injection needle attached or attachable to the syringe.

A “graded adverse event” refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention |
| | indicated; or limiting age-appropriate instrumental |
| | activities of daily living |
| 3 | Severe or medically significant, but not immediately life- |
| | threatening; hospitalization or prolongation of hospitalization |
| | indicated; disabling; or limiting self-care activities of daily |
| | living |
| 4 | Life-threatening consequences or urgent intervention indicated |
| 5 | Death related to adverse event |

### II. Combinations and Treatment Methods

The present invention concerns a method of treating an estrogen receptor-positive and HER2-positive breast cancer patient comprising administering to the patient a combination of pertuzumab, trastuzumab, and giredestrant in amounts effective to treat the breast cancer.

The present invention also concerns a method of treating an estrogen receptor-positive and HER2-positive breast cancer patient with advanced breast cancer comprising administering to the patient: a. induction therapy comprising four to eight cycles of a combination of pertuzumab and trastuzumab fixed dose combination (PH FDC) and docetaxel or paclitaxel in amounts effective to treat the breast cancer, followed by: b. maintenance therapy comprising a combination of giredestrant and PH FDC in amounts effective to treat the breast cancer.

The invention also provides a method of treating an estrogen receptor-positive and HER2-positive breast cancer patient with advanced breast cancer comprising administering to the patient a combination of pertuzumab and trastuzumab fixed dose combination (PH FDC), giredestrant, and palbociclib in amounts effective to treat the advanced breast cancer.

The invention additionally provides a method of treating an estrogen receptor-positive and HER2-positive breast cancer patient with advanced breast cancer comprising administering to the patient a combination of pertuzumab and trastuzumab fixed dose combination (PH FDC), giredestrant, and abemaciclib in amounts effective to treat the advanced breast cancer.

In particular embodiments of the above treatment methods:
the breast cancer is advanced breast cancer.
the breast cancer is locally advanced unresectable breast cancer.
the breast cancer is metastatic breast cancer.
the patient had prior disease progression on trastuzumab- and on HER2-ADC treatment (e.g. with ado-trastuzumab emtansine).
the breast cancer is early breast cancer and the combination is given as a neoadjuvant therapy or adjuvant therapy to treat the early breast cancer
the patient has a left ventricular ejection fraction (LVEF) of at least 50% measured by echocardiogram (ECHO) or multiple-gated acquisition scan (MUGA) prior to treatment (including prior to induction treatment and/or prior to maintenance treatment)
the pertuzumab and trastuzumab are administered as a fixed dose combination of pertuzumab and trastuzumab (PH FDC).
treatment with the combination pertuzumab+trastuzumab+giredestrant or PH FDC+giredestrant is more effective than treatment with pertuzumab and trastuzumab without giredestrant.

In one embodiment, the combination is administered as a maintenance therapy after induction therapy.

Induction therapy may comprise treatment of the patient with pertuzumab and trastuzumab and taxane, for example with 4 to 8 cycles of a combination of pertuzumab and trastuzumab fixed dose combination (PH FDC) and taxane selected from paclitaxel or docetaxel. The PH FDC and taxane are administered in amounts effective to treat the cancer. For example to achieve the minimum of stable disease (i.e. not progressive disease) prior to maintenance therapy.

According to such induction therapy including taxane chemotherapy:
paclitaxel is optionally administered as a 80 mg/m² dose intravenously on Day 1, Day 8, and Day 15 of each 21-day cycle; or
docetaxel is optionally administered as a 75 mg/m² dose intravenously on Day 1 of each 21-day cycle, with the docetaxel dose escalated to 100 mg/m² if the initial dose is well tolerated.

Maintenance therapy may comprise administering giredestrant together with pertuzumab and trastuzumab (e.g. as PH FDC) for one or more further cycles to further treat the breast cancer.

In one embodiment, giredestrant is administered at an amount of about 1 mg-100 mg, 1 mg-50 mg, 1 mg-30 mg, 10 mg-100 mg, 10 mg-50 mg, or 10 mg-30 mg every day. In another embodiment, giredestrant is administered at an amount of about 1, 5, 10, 15, 20, 25, 30, 50, or 100 mg. In still another embodiment, giredestrant is administered at an amount of about 10, 30, 50, or 100 mg. In still another embodiment, giredestrant is administered at an amount of 30 mg. In one embodiment, the giredestant is administered at a dose of 30 mg orally once daily on Days 1-21 of a 3-week or 4-week cycle.

In one embodiment, the patient is treated with the combination until disease progression or unacceptable toxicity.

The methods of treating breast cancer as provided herein can include administration of a combination therapy described herein as part of a dosing regimen. In one embodiment, the dosing regimen comprises one or more cycles. In another embodiment, the dosing regimen comprises at least 2 cycles. In another aspect provided herein is the dosing regimen comprises 1 to 200 cycles of treatment, e.g. 20 to 180 cycles, or 24 to 180 cycles. In one embodiment, the dosing regimen includes administration of a combination therapy as described herein in any number of cycles until the desired response (e.g. PFS, ORR, OS, DOR, DCR, CBR) reaches a desired outcome (e.g. increase in PFS, ORR, OS, DOR, DCR, CBR compared to a control described herein). In another embodiment, the dosing regimen includes administration of a combination therapy as described herein in any number of cycles until toxicity develops or the patient otherwise experiences one or more adverse events (AEs) that prevents further administration. In still another embodiment, the dosing regimen includes administration of a combination therapy as described herein in any number of cycles until disease progression.

In one embodiment, the maintenance therapy comprises administering giredestrant 30 mg orally once daily on Days 1-21 of a 21-day cycle in combination with PH FDC subcutaneously on Day 1 of a 21-day cycle.

The combination treatments, induction treatment, and maintenance treatment herein will be therapeutically effective. For example:
the treatment with the combination of giredestrant and PH FDC will achieve one or more clinical endpoint(s) selected from PFS, ORR, OS, DOR, DCR, and CBR (e.g. 1, 2, 3, 4, 5, 6 of these endpoints) greater than or superior to PH FDC alone;
the treatment with the combination of giredestrant and PH FDC will extend progression free survival (PFS) more than PH FDC alone;
the treatment with the combination of giredestrant and PH FDC increases median PFS from 4.5 months or more, or 6.7 months or more, compared with median PFS from PH FDC alone;
the treatment with the combination of giredestrant+PH FDC+abemaciclib or palbociclib will achieve one or more clinical endpoint(s) selected from PFS, ORR, OS, DOR, DCR, and CBR (e.g. 1, 2, 3, 4, 5, 6 of these endpoints) greater than or superior to PH FDC+giredestrant without abemaciclib or palbociclib; and/or
treatment with PH FDC+giredestrant+abemaciclib or palbociclib will achieve an greater ORR than that achieved with PH FDC+giredestrant without abemaciclib or palbociclib.

PH FDC can be subcutaneously administered as a loading dose FDC comprising 1200 mg pertuzumab and 600 mg trastuzumab (optionally further comprising 30,000 units hyaluronidase) followed by maintenance dose FDC comprising 600 mg pertuzumab and 600 mg trastuzumab (optionally further comprising 20,000 units hyaluronidase). Such PH FDC is optionally administered on Day 1 of a 3-week cycle.

In one embodiment, for patients receiving intravenous pertuzumab and trastuzumab with <6 weeks since their last dose, administer PH FDC as a maintenance dose of 600 mg pertuzumab and 600 mg trastuzumab and every 3 weeks for subsequent administrations.

In one embodiment, for patients receiving intravenous pertuzumab and trastuzumab with ≥6 weeks since their last dose, administer PH FDC as an initial dose of 1,200 mg pertuzumab and 600 mg trastuzumab, followed by a maintenance dose of 600 mg pertuzumab and 600 mg trastuzumab every 3 weeks for subsequent administrations.

In one embodiment, if dosing is paused and the time between PH FDC doses is greater than 6 weeks (and optionally 9 weeks or less), loading dose PH FDC (1200 mg pertuzumab+600 mg trastuzumab) should be given. Subsequent maintenance doses will be given every 3 weeks.

In one embodiment, the method comprises administering a FDC loading dose of pertuzumab, trastuzumab, and recombinant human hyaluronidase (rHuPH20) by subcutaneous injection in the thigh of the patient with a subcutaneous administration device (e.g. syringe) at a rate of about 2 mL/min over about 8 minutes. Optionally, the loading dose administration is followed by an about 30 minute observation period.

In one embodiment, the method optionally further comprises administering one or more FDC maintenance doses in the thigh of the patient at a rate of about 2 mL/min over about 5 min via a subcutaneous administration device. Optionally, administration of the maintenance doses are followed by an about 15 minute observation period, provided the loading dose was well tolerated.

For EBC, 2 to 10 (e.g. about 4) administrations of the FDC are given to the patient (e.g. as a neoadjuvant therapy prior to surgery) and, optionally, further post-surgery administrations, for example about 10 to 20 (e.g. about 18) maintenance administrations of the FDC are given to the patient following surgery, for example.

For advanced breast cancer (ABC), including locally advanced breast cancer (LABC) and metastatic breast cancer (MBC), the patient can be treated until disease progression or unacceptable toxicity. In one embodiment, from 1 to 200 maintenance doses, or 20 to 180 maintenance doses, or 24 to 180 maintenance doses are administered.

Optionally, abemaciclib is further administered to the patient. For example, abemaciclib can be administered 150 mg orally twice a day (during each 28-day cycle or 21-day cycle, depending on the regimen) until unacceptable toxicity or disease progression.

Optionally, palbociclib is further administered to the patient. For example Palbociclib can be administered at a dose of 125 mg orally every day Days 1-21 of each 28-day cycle. For example, palbociclib can be administered 125 mg orally once a day on Days 1-21 during each 28-day cycle until unacceptable toxicity or disease progression.

Exemplary doses administration modes, doses, and dosing regimens for the drugs for use herein include:

| Drug | Mode | Dose | Schedule |
|---|---|---|---|
| PH FDC | Subcutaneous | Loading/initial dose: | ABC induction therapy: every 3 weeks |
| | | 1200 mg P + 600 mg H | (q3w) for 4 to 8 cycles |
| | | | ABC maintenance therapy: q3w until |
| | | | disease progression and/or |
| | | | unacceptable toxicity |
| | | | EBC neoadjuvant therapy: q3w for 3 |
| | | Maintenance dose: | to 6 cycles |
| | | 600 mg P + 600 mg H | EBC adjuvant therapy: q3w for a total |
| | | | of 1 year (up to 18 cycles) or until |
| | | | disease recurrence or unmanageable |
| | | | toxicity |
| Giredestrant | Oral | 30 mg | Daily on days 1-21 of each 21-day |
| | | | cycle |
| | | | Daily on days 1-28 of each 28-day |
| | | | cycle (e.g. with palbociclib) |
| Palbociclib | Oral | 125 mg | Daily on days 1-21 of each 28-day |
| | | | cycle (with 7 days off) |
| Abemaciclib | Oral | 150 mg | Twice daily on days 1-21 of each 21- |
| | | | day cycle |

In one embodiment, the treatment excludes any additional endocrine therapy given concurrently.

In another embodiment, additional drugs or treatments that may further be combined with the treatments herein include, without limitation: endocrine therapy (e.g. tamoxifen or one of the specified third-generation AIs anastrozole, letrozole, or exemestane), LHRHa (e.g. leuprolide acetate, goserelin acetate, or triptorelin pamoate), bilateral oophorectomy, radiation therapy, and/or chemotherapy.

### III. Articles of Manufacture

In another embodiment of the invention, an article of manufacture containing materials useful for the treatment of cancer is provided. The article of manufacture comprises a subcutaneous administration device able to administer a FDC as disclosed herein by subcutaneous administration to a patient, for example a syringe, an injection device, an infusion pump, an injector pen, a needleless device, an autoinjector, and a subcutaneous patch delivery system.

In one embodiment, the device is a hand-held syringe, e.g. comprising a 25G-27G (⅜″-⅝″) hypodermic injection needle.

The subcutaneous administration device contains and delivers the FDC of the pertuzumab and trastuzumab, e.g. comprising approximately 600 mg or approximately 1200 mg of pertuzumab combined with approximately 600 mg of trastuzumab, and optionally further comprising 20,000 or 30,000 units of rHuPH20.

The article of manufacture preferably further comprises a package insert. The package insert may provide instructions to administer the FDC to a patient with HER2-positive, estrogen receptor-negative breast cancer, including locally advanced unresectable or metastatic breast cancer.

Another form of an article of manufacture is a syringe, containing the formulation to be administered, which may be attached to a stainless steel hypodermic needle for subcutaneous administration.

Optionally, the subcutaneous administration device comprises a 25G-27G (⅜″-⅝″) hypodermic hypodermic injection needle.

Optionally, the volume of the formulation in the subcutaneous administration device is adjusted to 15 mL for the initial or loading dose, and to 10 mL for the subsequent or maintenance doses.

In one embodiment, the article of manufacture comprises two vials, wherein a first vial contains loading dose FDC (e.g. comprising 1200 mg pertuzumab, 600 mg trastuzumab, 30,000 units of rHuPH20, e.g. total volume of about 15 mL), and a second vial contains a maintenance dose FDC (e.g. comprising 600 mg pertuzumab, 600 mg trastuzumab, 30,000 units of rHuPH20, e.g. total volume of about 10 mL).

Further details of the invention are illustrated by the following non-limiting Examples. The disclosures of all citations in the specification are expressly incorporated herein by reference.

### Example 1

Assessing the Anti-Proliferative Activity of Giredestrant in Combination with Trastuzumab and Pertuzumab in ER+/HER2+ Breast Cancer Cell Lines

Background: HER2 amplification is proposed to be a key resistance mechanism limiting the activity of endocrine therapies in ER+/HER2+ breast cancer. This experiment evaluated whether the anti-proliferative activity of the ER antagonist and degrader, giredestrant, was enhanced in the presence of HER2-targeted therapy, trastuzumab and pertuzumab, in ER+/HER2+ breast cancer cell lines.

Materials and Methods: ER+/HER2+ cell lines UACC-812, HCC1419 and ZR-75-30 cells were plated in 50 μL culture media in 384-well plates. Cells were incubated in humidified incubator overnight (37 degree, 5% CO2). Test materials were dispensed into 384-well plate on day 1; giredestrant was evaluated as a 9-point dose response (3-fold dose dilutions from a maximum concentration of 300 nM), and traztuzumab and pertuzumab were evaluated as a fixed dose at 30 μg/ml each. Fluorescence-based cell proliferation (CyQUANT®; ThermoFisher Scientific, Cat #C7026) readings were taken at day 1 to establish a baseline, and at 6 days following drug treatment. Analysis of the drug response was performed using the growth-rate (GR) inhibition method to avoid biases between slow and fast-growing lines (Hafner et al., Nat Methods 2016 June; 13 (6): 521-7)). The growth rate inhibition value of cells under control conditions is set to 1 (GR=1), and a lack of net gain in cell numbers is set to 0 (GR=0).

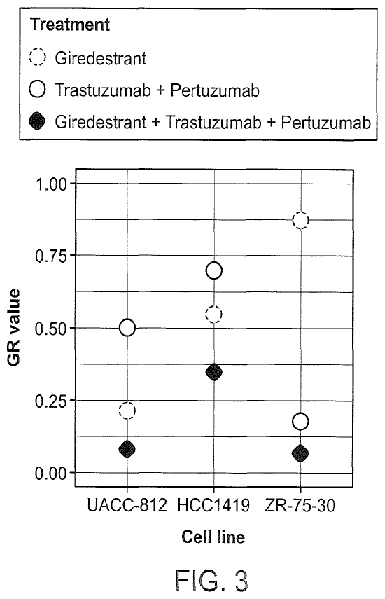

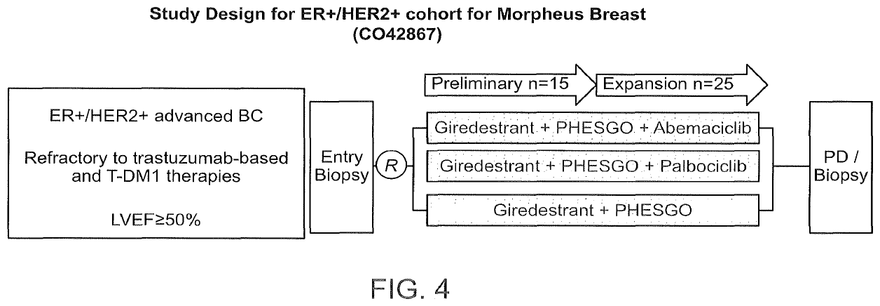

Results: Giredestrant displays meaningful single agent anti-proliferative activity in UACC-812 and HCC1419 cells (GR<0.5), and more modest activity in ZR-75-30 cells (GR>0.75). In contrast, ZR-75-30 cells display greatest sensitivity to trastuzumab and pertuzumab. For each of the three cell lines, GR values are lowest when giredestrant, trastuzumab and pertuzumab are combined (see FIG. 3).

Conclusion: The combined inhibition of ER by giredestrant and HER2 suppression by trastuzumab and pertuzumab leads to a more profound anti-proliferative effect than either ER or HER2 suppression alone, in UACC-812, HCC1419 and ZR-75-30.

### Example 2

### Giredestrant+PH FDC±CDK4/6 Inhibitor Combination Therapy

This study will evaluate the efficacy, safety, and pharmacokinetics of giredestrant and fixed dose combination of pertuzumab and trastuzumab (PH FDC) for subcutaneous administration as well as triplet combinations of giredestrant and PH FDC SC in combination with either abemaciclib (Gire+PH FDC SC+Abema) or palbociclib (Gire+PH FDC SC+Palbo) in patients with inoperable, locally advanced unresectable or metastatic ER+, HER2-positive breast cancer with prior disease progression on trastuzumab- and ado-trastuzumab emtansine-based therapies.

### Giredestrant Plus PH FDC SC

Patients in the giredestrant plus PH FDC SC arm will receive treatment as outlined in Table 1 until unacceptable toxicity or disease progression as determined by the investigator according to RECIST v1.1.

TABLE 1
Treatment Regimen for Gire + PH FDC SC Arm

| Cycle Length | Dose, Route, and Regimen |
|---|---|
| 21 days | Giredestrant: 30 mg PO QD during each 21-day cycle |
| | PH FDC SC: |
| | Loading dose: 1200 mg pertuzumab, 600 mg trastuzumab, and |
| | 30,000 units hyaluronidasc SC on Day 1 of Cycle 1 ^{a} |
| | Maintenance doses: 600 mg pertuzumab, 600 mg trastuzumab, and |
| | 20,000 units hyaluronidase SC on Day 1 of Cycles 2 and beyond ^{b} |

Gire = giredestrant; PO = by mouth; PH FDC SC = fixed-dose combination of pertuzumab and trastuzumab for subcutaneous administration; QD = once a day.
^{a} Loading dose will be administered subcutaneously over approximately 8 minutes.
^{b} Maintenance doses will be administered subcutaneously over approximately 5 minutes.

### Giredestrant Plus PH FDC SC Plus Abemaciclib

Patients in the giredestrant plus PH FDC SC plus abemaciclib arm will receive treatment as outlined in Table 2 until unacceptable toxicity or disease progression as determined by the investigator according to RECIST v1.1.

TABLE 2
Treatment Regimen for Gire + PH FDC SC + Abema Arm

| Cycle Length | Dose, Route, and Regimen |
|---|---|
| 21 days | Giredestrant: 30 mg PO QD during each 21-day cycle |
| | Abemaciclib: 150 mg PO BID during each 21-day cycle |
| | PH FDC SC: |
| | Loading dose: 1200 mg pertuzumab, 600 mg trastuzumab, and |
| | 30,000 units hyaluronidase SC on Day 1 of Cycle 1 ^{a} |
| | Maintenance doses: 600 mg pertuzumab, 600 mg trastuzumab, and |
| | 20,000 units hyaluronidase SC on Day 1 of Cycles 2 and beyond ^{b} |

Abema = abemaciclib; Gire = giredestrant; PO = by mouth; BID = twice a day; PH FDC SC = fixed-dose combination of pertuzumab and trastuzumab for subcutaneous administration; QD = once a day.
^{a} Loading dose will be administered subcutaneously over approximately 8 minutes.
^{b} Maintenance doses will be administered subcutaneously over approximately 5 minutes.

Giredestrant plus PH FDC SC plus Palbociclib

Patients in the giredestrant plus PH FDC SC plus palbociclib arm will receive treatment as outlined in Table 3 until unacceptable toxicity or disease progression as determined by the investigator according to RECIST v1.1.

TABLE 3
Treatment Regimen for Gire + PH FDC SC + Palbo Arm

| Cycle Length | Dose, Route, and Regimen |
|---|---|
| 28 days | Palbociclib: 125 mg PO daily for Day 1-21 during each 28-day cycle |
| | Giredestrant: 30 mg PO QD for Day 1-28 during each 28-day cycle |
| 21 days | PH FDC SC: |
| | Loading dose: 1200 mg pertuzumab, 600 mg trastuzumab, and |
| | 30,000 units hyaluronidase SC on Day 1 of Cycle 1 ^{a} |
| | Maintenance doses: 600 mg pertuzumab, 600 mg trastuzumab, and |
| | 20,000 units hyaluronidase SC on Day 1 of Cycles 2 and beyond ^{b} |

Gire = giredestrant; Palbo = palbociclib; PO = by mouth; PH FDC SC = fixed-dose combination of pertuzumab and trastuzumab for subcutaneous administration; QD = once a day.
^{a} Loading dose will be administered subcutaneously over approximately 8 minutes.
^{b} Maintenance doses will be administered subcutaneously over approximately 5 minutes.

TABLE 4
Primary, Secondary, and Safety Endpoints of the Trial

| Primary Efficacy Objective | Corresponding Endpoint |
|---|---|
| Efficacy of the combination | ORR, defined as the proportion of patients with |
| | a complete response or partial response, as |
| | determined by the investigator according to |
| | RECIST v1.1 |

| | Corresponding Endpoints |
|---|---|
| Secondary Efficacy Objective | |
| Efficacy of the combination | PFS, defined as the time from randomization to |
| | the date of the first recorded occurrence of |
| | disease progression or death from any cause |
| | (whichever occurs first) in Stage 1, as |
| | determined by the investigator according to |
| | RECIST v1.1 |
| | DCR, defined as proportion of patients with |
| | stable discase for ≥12 weeks or a complete or |
| | partial response, as determined by the |
| | investigator according to RECIST v1.1 |
| | CBR, defined as proportion of patients with |
| | stable disease ≥24 weeks or with confirmed |
| | complete or partial response, as determined by |
| | the investigator according to RECIST v1.1 |
| | OS, defined as the time from randomization to |
| | death from any cause |
| | DOR, defined as the time from the first |
| | occurrence of a documented OR to the first date |
| | of recorded disease progression or death from |
| | any cause (whichever occurs first), as |
| | determined by the investigator according to |
| | RECIST v1.1 |
| Safety Objective | |
| Safety of the combination | Incidence, nature, and severity of adverse events |
| | and laboratory abnormalities, with severity |
| | determined according to NCI CTCAE v5.0 |

### Patients Treated in the Study

1. Female
2. ECOG Performance Status of 0 or 1
3. Histologically or cytologically confirmed and documented adenocarcinoma of the breast with metastatic or locally advanced unresectable disease not amenable to curative resection.
4. HER2-positive as defined by ASCO/CAP guidelines:
   a. >10% of contiguous and homogenous tumor cells showing protein overexpression (3+) on circumferential membrane staining that is complete and intense by IHC
   b. ISH positive based on counting at least 20 cells within the area of >10% contiguous and homogenous tumor cells showing:
      Single-probe average HER2 copy number≥6.0 signals/cell.
      Dual-probe HER2/CEP17 ratio≥2.0, with an average HER2 copy number≥4.0 signals/cell
      Dual-probe HER2/CEP17 ratio≥2.0, with an average HER2 copy number<4.0 signals/cell (in rare cases such as chromosome 17 monosomy)
      Dual-probe HER2/CEP17 ratio<2.0, with an average HER2 copy number≥6.0 signals/cell
5. Documented ER+tumor according to ASCO/CAP guidelines, assessed locally and defined as ≥1% of tumor cells stained positive based on the most recent tumor biopsy (or archived tumor sample)
6. Patient must be considered appropriate for ET and anti-HER2 therapy.
7. Previous progression to standard of care anti-HER2 therapies, of which one was a trastuzumab-and-taxane-based systemic therapy (including in the early setting if recurrence occurred within 6 months of finishing adjuvant therapy) and one was ado-trastuzumab emtansine
8. Up to one line of ET in the advanced setting allowed, including fulvestrant if given more than 28 days prior to randomization, but excluding other SERDs.
9. Measurable disease (at least one target lesion) according to RECIST v1.1
10. Baseline LVEF≥50% as measured by ECHO or MUGA scans

### Efficacy and Safety Outcomes

The treatment with the combination of giredestrant+PH FDC will be effective according to any one or more of the primary and secondary endpoints, and will have acceptable safety, in patients with locally advanced unresectable or metastatic estrogen receptor-positive, HER2-positive breast cancer.

The treatment with the combination of giredestrant+PH FDC+abemaciclib will be more effective than the treatment with giredestrant+PH FDC without abemaciclib according to any one or more of the primary and secondary endpoints, and will have acceptable safety, in patients with locally advanced unresectable or metastatic estrogen receptor-positive, HER2-positive breast cancer.

The treatment with the combination of giredestrant+PH FDC+palbociclib will be more effective than the treatment with giredestrant and PH FDC without palbociclib according to any one or more of the primary and secondary endpoints, and will have acceptable safety, in patients with locally advanced unresectable or metastatic estrogen receptor-positive, HER2-positive breast cancer.

### Example 3

Giredestrant in Combination with Pertuzumab and Trastuzumab Fixed Dose Combination (PH FDC) Versus PH FDC after Induction Therapy with PH FDC Plus Taxane in Patients with HER2-Positive Estrogen Receptor-Positive Advanced Breast Cancer

This study will assess the efficacy and safety of giredestrant, a novel oral selective estrogen receptor degrader (SERD) in combination with PHESGOR (fixed dose combination pertuzumab, trastuzumab, and rHuPH20 injection, for SC use) in participants with previously untreated, locally-advanced unresectable, or metastatic, estrogen receptor (ER)-positive, HER2-positive breast cancer (BC), following four to eight cycles of induction therapy with PH FDC+taxane (i.e., docetaxel or paclitaxel, as per the standard of care). Despite advances in early diagnosis and curative multimodality treatments, some patients may still experience a metastatic recurrence or present with “de novo” metastatic breast cancer (MBC). There continues to be a need for treatments with better benefit/risk profiles that prolong progression free survival (PFS) and other survival endpoints of patients with ER-positive, HER2-positive advanced breast cancer (ABC).

The primary comparison of interest is the hazard ratio (HR) of PFS. The primary trial objective is to demonstrate superiority of the giredestrant plus PH FDC arm over the PH FDC arm.

In this protocol, “induction therapy” refers to treatment with PH FDC+taxane and “maintenance treatment” refers to PH FDC plus giredestrant or PH FDC.

This Phase III, randomized, two-arm, open-label, multicenter study will evaluate the efficacy and safety of PH FDC plus giredestrant compared with PH FDC after induction with PH FDC+taxane in participants with HER2-positive, ER-positive advanced breast cancer (metastatic or locally-advanced disease not amenable to curative treatment) who have not previously received a systemic non-hormonal anti-cancer therapy in the advanced setting.

During the induction therapy phase, participants will receive four to six cycles of PH FDC in combination with a taxane (i.e., docetaxel or paclitaxel, as per the standard of care). At the investigator's discretion, participants who tolerate six cycles of induction therapy well and do not experience progressive disease (PD) may be given up to two additional cycles: up to a maximum of eight cycles as per the standard of care. Participants who have received one or two cycles of PH FDC (or trastuzumab SC with pertuzumab IV, or PH IV) with docetaxel or paclitaxel prior to enrollment are eligible and these additional cycles will count towards eligibility for the maintenance phase.

Following the induction therapy phase, eligible participants will be randomized into the maintenance therapy phase during which they will receive PH FDC plus giredestrant or PH FDC in 21-day cycles until discasc progression, unacceptable toxicity, withdrawal of consent, death, or predefined study end, whichever occurs first.

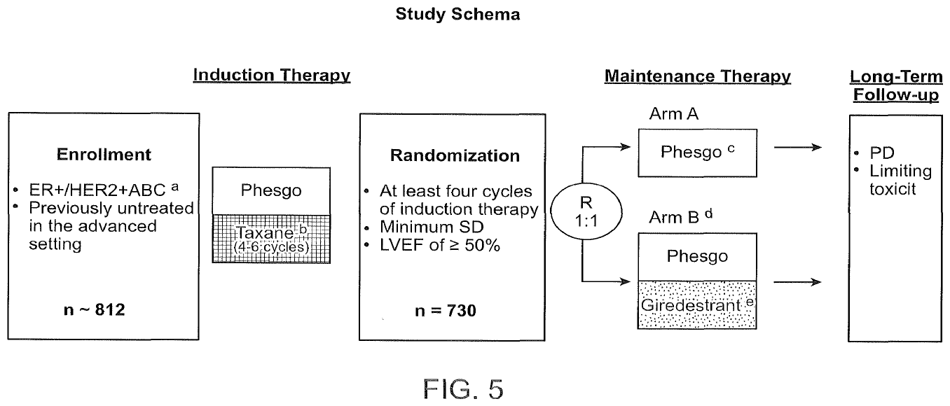

A study schema is provided in FIG. 5 and the endpoints are in Table 5.

TABLE 5
Primary, Secondary, and Safety Endpoints of the Trial

| Primary Objective | Corresponding Endpoint |
|---|---|
| Efficacy of PH FDC | PFS, defined as the time from randomization to the first |
| plus giredestrant | occurrence of disease progression or death from any cause |
| compared with PH FDC | (whichever occurs first), as determined by the investigator |
| | according to RECIST v1.1 |

| Secondary Objectives | Corresponding Endpoints |
|---|---|
| Efficacy of PH FDC plus | OS, defined as the time from randomization to death from any |
| giredestrant compared | cause |
| with PH FDC | ORR (following randomization), defined as the proportion of |
| | participants with a CR or PR on two consecutive occasions ≥ |
| | 4 weeks apart, as determined by the investigator according to |
| | RECIST v1.1 |
| | DOR (following randomization), defined as the time from the |
| | first occurrence of a documented objective response to disease |
| | progression or death from any cause (whichever occurs first), as |
| | determined by the investigator according to RECIST v1.1 |
| | CBR (following randomization), defined as the proportion of |
| | participants with SD for ≥24 weeks or a CR or PR, as |
| | determined by the investigator according to RECIST v1.1 |
| | Mean and mean changes from baseline score in function (role, |
| | physical) and HRQOL by cycle and between treatment arms as |
| | assessed through the use of the Functional and GHS/QOL scales |
| | of the EORTC QLQ-C30 |
| Safety of PH FDC plus | Incidence and severity of adverse events, with severity |
| giredestrant compared | determined according to NCI CTCAE v5.0 |
| with PH FDC | Change from baseline in targeted clinical laboratory test results |

CBR = clinical benefit rate; CR = complete response; CTCAE = Common Terminology Criteria for Adverse Events; DOR = duration of response; GHS/QoL = global health status/ quality of life; HRQol = health-related quality of life; PFS = progression-free survival; PR = partial response; NCI = National Cancer Institute; ORR = objective response rate; OS = overall survival; RECIST v1.1 = Response Evaluation Criteria in Solid Tumors, Version 1.1; SD = stable disease.

### Induction Therapy Phase

During the induction therapy phase, all participants will receive PH FDC in combination with a taxane (i.e., docetaxel or paclitaxel) for four to six cycles, as per the standard of care.

At the investigator's discretion, participants who tolerate six cycles of induction therapy well and in the absence of PD or limiting toxicity, may be given up to two additional cycles of the same combination taxane+PH FDC, for a total of up to eight cycles.

Participants who have received one or two cycles of PH FDC (or pertuzumab SC with trastuzumab IV, or PH IV) with docetaxel or paclitaxel prior to enrollment are eligible, provided they have not experienced PD or limiting toxicity. Any off-study cycles will count towards the four to eight cycles allowed and eligibility for the maintenance phase.

Participants who are unable to tolerate the assigned taxane and discontinue prior to the minimum of four cycles will discontinue all study treatment and enter the follow-up phase. The participant will be treated as per the standard of care at the discretion of the investigator, as clinically indicated.

### Inclusion Criteria

1. Histologically or cytologically confirmed and documented adenocarcinoma of the breast with metastatic or locally-advanced disease not amenable to curative resection.
2. HER2-positive ABC confirmed by a central laboratory prior to study enrollment. HER2-positive status will be determined based on primary or metastatic lesion and defined as 3+ by immunohistochemistry (IHC) and/or positive by HER2 amplification by in situ hybridization (ISH) with a ratio of ≥2 for the number of HER2 gene copies to the number of signals for chromosome 17 copies.
3. ER-positive tumor according to the American Society of Clinical Oncology/College of American Pathologists (ASCO/CAP) guidelines, assessed locally and defined as ≥1% of tumor cells staining positive for ER, preferentially based on the same lesion that was used to determine HER2 positivity.
4. At least one measurable lesion and/or non-measurable disease evaluable according to RECIST version 1.1
5. Disease-free interval from completion of adjuvant or neoadjuvant systemic non-hormonal treatment to recurrence of ≥6 months
6. Eastern Cooperative Oncology Group (ECOG) Performance Status 0 or 1.
7. LVEF of at least 50% measured by echocardiogram (ECHO) or multiple-gated acquisition scan (MUGA).

### Maintenance Therapy Phase

Following the induction therapy phase, participants who meet the following criteria will be eligible for the maintenance phase:
1. Complete a minimum of four cycles of induction therapy, defined as either:
   4 PH FDC injections+4 docetaxel infusions or
   4 PH FDC injections+12 paclitaxel infusions
      Note: If a participant has received one or two cycles of induction therapy prior to enrollment, these cycles are to be counted towards the required number of cycles for cligibility of the maintenance phase (e.g., if the participant was given onc cycle of PH FDC [or trastuzumab SC with pertuzumab IV, or PH IV] +docetaxel prior to enrollment, a minimum of 3 on-study cycles of PH FDC+docetaxel are required prior to entering the maintenance phase)
2. Achieve a minimum of stable disease (SD) (i.e., did not experience progressive disease [PD]) according to RECIST v1.1 at the last tumor assessment during the induction therapy phase
3. Left ventricular ejection fraction (LVEF) of ≥50% at the last assessment during the induction therapy phase.

Eligible participants will be randomized in a 1:1 ratio to one of two treatment arms:Crossover between the two treatment arms will not be allowed. Prior to randomizing a participant, investigators must decide and document if optional ET will be prescribed according to the standard of care, in the event that the participant is randomized to Arm A.
Arm A (PH FDC: control arm): Participants will receive PH FDC subcutaneously every 3 weeks (Q3W). Optional concomitant ET of investigator's choice is allowed based on the standard of care (ETs can include an AI or tamoxifen). Pre- and perimenopausal women or men who receive an AI must also receive a luteinizing hormone-releasing hormone agonist (LHRHa). Alternatively, women who are pre- or perimenopausal can be treated with bilateral oophorectomy.
Arm B (PH FDC plus giredestrant: experimental arm): Participants will receive giredestrant 30 mg orally (PO) once daily (QD) on Days 1-21 of each 21-day cycle in combination with PH FDC subcutaneously Q3W. Pre- and perimenopausal women, and all men will receive a LHRHa every 28 days and up to 28 days prior to the first giredestrant dose. Alternatively, pre- or perimenopausal women can be treated with bilateral oophorectomy.
   Table 6 provides a description of assigned study treatments for this study.

TABLE 6
Study Treatment Description

| | PH FDC | Giredestrant |
|---|---|---|
| Use | Experimental | Experimental |
| Drug form | Solution for injection | Capsule |
| Unit Dose Strength(s) | Loading: | 30 mg/capsule |
| | pertuzumab 80 mg/mL, | |
| | trastuzumab 60 mg/mL, | |
| | rHuPH20 2,000 U/mL | |
| | Maintenance: | |
| | pertuzumab 60 mg/mL, | |
| | trastuzumab 60 mg/mL, | |
| | rHuPH20 2,000 U/mL | |
| Dosage Level(s) | Loading: | 30 mg QD Day 1-21 of each 21 day |
| | pertuzumab 1200 mg | cycle |
| | trastuzumab 600 mg | |
| | rHuPH20 30,000 U | |
| | Maintenance: | |
| | pertuzumab 600 mg | |
| | trastuzumab 600 mg | |
| | rHuPH20 20,000 U | |
| | Q3W | |
| Formulation(s) | Refer to pharmacy manual and/or | Refer to pharmacy manual and/or |
| | Investigator's Brochure | Investigator's Brochure |
| Packaging | Loading: 20 mL glass vials | HDPE bottles with a plastic child- |
| | Maintenance: 15 mL glass vials | resistant cap with induction seal |
| | | and desiccant |
| Route of | SC injection | Oral |
| administration | | |
| Source | Sponsor | Sponsor |

HDPE = high-density polyethylene; Q3W = every 3 weeks; QD = every day.

At applicable sites, during the maintenance phase study treatment may be administered by a trained nursing professional at the participant's home or another suitable location, if the participant has given written informed consent to participate in mobile nursing (MN) visits and if this is possible per country regulations.

### PH FDC

PH FDC will be provided in single-dose, ready-to-use glass vials and administered subcutaneously as a fixed non-weight-based dose. During the induction therapy phase, PH FDC will be administered prior to taxane-based chemotherapy (i.e., docetaxel or paclitaxel). If given prior to enrollment, the same taxane used outside the trial should be administered during the induction therapy phase. Participants who experience injection-related symptoms may be pre-medicated with analgesics and antihistamines prior to subsequent injections.

In the induction therapy phase, a loading dose (1200 mg pertuzumab, 600 mg trastuzumab, 30,000 U rHuPH20) will be administered in the first cycle. In subsequent cycles, maintenance doses (600 mg pertuzumab, 600 mg trastuzumab, 20,000 U rHuPH20) will be administered subcutaneously Q3W. If a participant was administered one or two doses of PH FDC (or trastuzumab SC plus pertuzumab IV, or trastuzumab IV plus pertuzumab) less than 6 weeks prior to enrollment in the induction phase, PH FDC maintenance dose will be administered. However, if dosing is paused and the time between PH FDC doses is ≤6 weeks but≤9 weeks, a loading dose should be given. Subsequent maintenance doses will be given Q3W. No dose reductions are allowed for PH FDC.

PH FDC will be administered in accordance with prescribing information. All doses of PH FDC will be administered over 5-8 minutes as a SC injection into the thigh (no other anatomical location is allowed) at a rate of no more than 2 mL/min. Loading dose(s) should be administered over 8 minutes; maintenance doses should be administered over 5 minutes. The injection rate should be adjusted to a rate that is comfortable for the participant. New injections should be given at least 2.5 cm from the previous site and never into areas where the skin is red, bruised, tender, or hard. The entire volume (15 mL volume for the loading dose; 10 mL volume for the maintenance dose) must be injected in one site: splitting the volume into two syringes or injecting at two different sites is not permitted.

After the first loading dose injection, participants will be observed for injection-related symptoms for 30 minutes following the end of the injection. If the participant experiences injection-related symptoms during the injection, the injection should be slowed or interrupted (but may not be reduced). If the first injection is well tolerated, participants will be observed for 15 minutes following subsequent injections.

### Giredestrant

Giredestrant will be supplied as an immediate-release capsule, packaged in high-density polyethylene bottles with a plastic child-resistant cap with induction seal and desiccant. On Days 1-21 of each 21-day cycle during the maintenance treatment phase, participants who have been randomized to Arm B will self-administer one 30-mg giredestrant capsule orally at approximately the same time each day. Giredestrant may be taken with or without a meal.

For cycles where PH FDC is administered in the clinic, the Day 1 dose of giredestrant will be administered in the clinic. For any cycles where PH FDC is administered outside of the clinic (at the participant's home or other suitable location), the Day 1 giredestrant dose may also be administered outside of the clinic. If a dose is missed it should be made up, unless the next dose is due within 6 hours. If a dose is vomited, the participant should resume dosing with the next scheduled dose; vomited doses will not be made up.

If giredestrant is withheld or needs to be permanently discontinued for treatment-related toxicity, the participant should continue to receive treatment with PH FDC alone.

### Taxane

During the induction therapy phase, the investigator's choice of taxane-based chemotherapy (i.e., docetaxel or paclitaxel) will be administered after PH FDC. If a participant received a taxane prior to enrollment, the same taxane used outside the trial should be administered during the induction therapy phase. Refer to the currently approved prescribing information for docetaxel and paclitaxel, as applicable, for formulation, handling, and dosing instructions.

A recommended dosing scheduled is provided in Table 7.

TABLE 7
Recommended Dosing Schedule for Docetaxel and Paclitaxel

| Taxane | Schedule |
|---|---|
| Docetaxel | Administer 75 mg/m² intravenously over 60 (±10) minutes |
| | on Dl of cach cycle for 4 to 8 cycles (a cycle is 21 days); |
| | this dose may be escalated to 100 mg/m² if the initial dose |
| | was well tolerated |
| Paclitaxel | Administer 80 mg/m² intravenously over a minimum of |
| | 1 hour on D 1, D 8, D 15 of each cycle for 4 to 8 cycles (a |
| | cycle is 21 days); this weekly regimen is considered as one |
| | complete cycle whenever 3 weekly doses are given |

### Optional Endocrine Therapy of Investigator's Choice

Optional ET are: tamoxifen or one of the specified third-generation AIs (anastrozole, letrozole, or exemestane). Dose administration of ET should be performed in accordance with the local prescribing information for the respective product. If the choice of ET needs to be permanently discontinued for treatment-related toxicity, ET must be permanently discontinued and the participant should continue to receive treatment with PH FDC alone.

### Luteinizing Hormone-Releasing Hormone Agonist

LHRHa, which may include, but are not limited to, leuprolide acetate, goserelin acetate, or triptorelin pamoate, will be administered to male participants, and pre- and perimenopausal female participants while receiving giredestrant in Arm B. LHRHa may be administered to male and pre- and perimenopausal female participants receiving tamoxifen in Arm A, and should be administered to those receiving an AI in Arm A.

The investigator will determine and supply the appropriate LHRHa locally approved for use in BC. LHRHa will be administered according to local prescribing information. Monthly injections are preferred to minimize the potential of exposure to the medication decreasing to sub-therapeutic levels towards the end of the treatment cycle.

If the participant becomes intolerant to current LHRHa, the participant may switch to another approved LHRHa during the study. Bilateral oophorectomy for pre- or perimenopausal women is allowed.

### Treatment Outcomes

Treatment with the combination of giredestrant and the PH FDC will achieve any one or more of the efficacy endpoints superior to PH FDC alone, with acceptable toxicity. For example, treatment with the combination will extend progression free survival (PFS) more than PH FDC alone, with acceptable toxicity.

In one embodiment, treatment with giredestrant and the PH FDC will extend the median progression free survival (PFS) from randomization (i.e. from the start of maintenance therapy) by 4.5 months or more, or by 6.7 months or more, compared to median PFS with PH FDC alone.

What is claimed is:

1. A method of treating an estrogen receptor-positive and HER2-positive breast cancer patient comprising administering to the patient a combination of pertuzumab, trastuzumab, and giredestrant in amounts effective to treat the breast cancer.

2. The method of claim 1, wherein the pertuzumab and trastuzumab are administered as a fixed dose combination of pertuzumab and trastuzumab (PH FDC).

3. The method of claim 1 or claim 2, wherein the breast cancer is advanced breast cancer.

4. The method of claim 4, wherein the advanced breast cancer is locally advanced unresectable breast cancer or metastatic breast cancer.

5. The method of claim 1, wherein treatment with the combination is more effective than treatment with pertuzumab and trastuzumab without giredestrant.

6. The method of claim 1, wherein the combination is administered as a maintenance therapy after induction therapy.

7. The method of claim 6, wherein the induction therapy comprises treatment of the patient with pertuzumab and trastuzumab and taxane.

8. The method of claim 7, wherein the induction therapy comprises 4 to 8 cycles of a combination of pertuzumab and trastuzumab fixed dose combination (PH FDC) and paclitaxel or docetaxel in amounts effective to treat the breast cancer.

9. The method of claim 8, wherein the paclitaxel is administered as a 80 mg/m² dose intravenously on Day 1, Day 8, and Day 15 of each 21-day cycle; and docetaxel is administered as a 75 mg/m² dose intravenously on Day 1 of each 21-day cycle, wherein the docetaxel dose is escalated to 100 mg/m² if the initial dose is well tolerated.

10. The method of claim 6, wherein the maintenance therapy comprises administering giredestrant 30 mg orally once daily on Days 1-21 of a 21-day cycle in combination with PH FDC subcutaneously on Day 1 of a 21-day cycle.

11. The method of claim 2, wherein the treatment with the combination of giredestrant and PH FDC extends progression free survival (PFS) more than PH FDC alone.

12. The method of claim 11, wherein the treatment with the combination of giredestrant and PH FDC increases median PFS from 4.5 months or more compared with median PFS from PH FDC alone.

13. The method of claim 2, wherein the PH FDC is subcutaneously administered as a loading dose FDC comprising 1200 mg pertuzumab and 600 mg trastuzumab followed by maintenance dose FDC comprising 600 mg pertuzumab and 600 mg trastuzumab.

14. The method of claim 13, wherein the PH FDC is administered on Day 1 of a 3-week cycle.

15. The method of claim 13 or claim 14, wherein the loading dose FDC further comprises 30,000 units hyaluronidase and the maintenance dose FDC further comprises 20,000 units hyaluronidase.

16. The method of any one of the preceding claims, wherein the giredestant is administered at a dose of 30 mg orally once daily for a 3-week or 4-week cycle.

17. The method of any one of the preceding claims, further comprising administering a CDK4/6 inhibitor to the patient.

18. The method of claim 17, wherein the CDK4/6 inhibitor comprises palbociclib.

19. The method of claim 18, wherein the palbociclib is administered at a dose of 125 mg orally daily for Day 1-21 during a 28-day cycle.

20. The method of claim 17, wherein the CDK4/6 inhibitor comprises abemaciclib.

21. The method of claim 20, wherein the abemaciclib is administered at a dose of 150 mg orally twice a day during a 21-day cycle.

22. The method of any one of the preceding claims, wherein the patient had prior disease progression on trastuzumab treatment and on HER2-ADC treatment.

23. A method of treating an estrogen receptor-positive and HER2-positive breast cancer patient with advanced breast cancer comprising administering to the patient:
a. induction therapy comprising four to eight cycles of a combination of pertuzumab and trastuzumab fixed dose combination (PH FDC) and docetaxel or paclitaxel in amounts effective to treat the breast cancer, followed by:
b. maintenance therapy comprising a combination of giredestrant and PH FDC in amounts effective to treat the breast cancer.

24. A method of treating an estrogen receptor-positive and HER2-positive breast cancer patient with advanced breast cancer comprising administering to the patient a combination of pertuzumab and trastuzumab fixed dose combination (PH FDC), giredestrant, and palbociclib in amounts effective to treat the advanced breast cancer.

25. A method of treating an estrogen receptor-positive and HER2-positive breast cancer patient with advanced breast cancer comprising administering to the patient a combination of pertuzumab and trastuzumab fixed dose combination (PH FDC), giredestrant, and abemaciclib in amounts effective to treat the advanced breast cancer.
